(19) United States
(12) Patent Application Publication
(10) Pub. No.: US 2025/0205285 A1
(43) Pub. Date: Jun. 26, 2025

(54) COMPOSITION FOR ORAL DAMAGE MITIGATION, USE THEREOF, AND MANUFACTURING METHOD THEREOF

(71) Applicant: TAIWAN MITOCHONDRION APPLIED TECHNOLOGY CO., LTD., Zhubei City, Hsinchu County (TW)

(72) Inventors: Han-Chung CHENG, Zhubei City (TW); Hui-Ching TSENG, Zhubei City (TW); Chih-Kai HSU, Zhubei City (TW); Li-Hsin YAO, Zhubei City (TW); Tsung-You YAO, Zhubei City (TW); Cheng-Wei WU, Zhubei City (TW)

(21) Appl. No.: 18/844,740

(22) PCT Filed: May 16, 2023
(86) PCT No.: PCT/CN2023/094569

§ 371 Date: Sep. 6, 2024

Related U.S. Application Data
(60) Provisional application No. 63/342,568, filed on May 16, 2022.

(51) Int. Cl. A61K 35/28; A61K 35/19; A61P 1/02

(52) U.S. Cl. CPC A61K 35/28; A61K 35/19; A61P 1/02

(57) ABSTRACT

A composition for oral damage mitigation, use thereof, and manufacturing method thereof are disclosed. The composition includes mitochondria and a biocompatible carrier. The composition can mitigate, repair, ameliorate, or treat oral damage and can be expected to be a composition or medicament that is able to mitigate, repair, ameliorate, or treat periodontal diseases or oral cancers while having both safety and effectiveness.

### CROSS-REFERENCE TO RELATED APPLICATIONS

This application is a U.S. National Phase of International Application No. PCT/CN2023/094569, filed on May 16, 2023, which claims the benefit of U.S. provisional patent application No. 63/342,568, filed on May 16, 2022, the entire contents of which are hereby incorporated by reference.

### BACKGROUND

### 1. Technical Field

The present disclosure relates to a composition for oral damage mitigation, use thereof, and manufacturing method thereof.

### 2. Related Art

Gingival fibroblasts are the primary cells in the periodontium tissue. When the periodontium tissue is damaged, the gingival fibroblasts regulate various cell functions such as proliferation, migration, elongation, and adhesion, thereby remodeling the tissue and regulating wound healing to maintain the homeostasis of the periodontium tissue to support and stabilize the teeth. Periodontal diseases refer to any diseases related to the tissues surrounding and supporting the teeth. The periodontal diseases include gingivitis and periodontitis, the former can be reversed with proper oral hygiene and treatment, while the latter is an irreversible condition. Prolonged inflammation of the gums can damage the periodontium tissue, leading to the progression from gingivitis to periodontitis.

Studies have shown that air pollution is also associated with periodontal disease, periodontal abscess, oral submucous fibrosis, Leukoplakia, and oral cancer. Air pollutants include carbon monoxide, sulfur and nitrogen oxides, ozone, and particulate matters (PM). The particulate matters include various substances such as ions, metals, ammonium salts, sulfates, nitrates, carbon, organic carbon compounds, and silica, with varying compositions and some being water-soluble. The particulate matters originate not only from human-made pollution but also from natural sources. Recently, evidences have shown that the particulate matters are associated with respiratory diseases, cardiovascular diseases, cerebrovascular diseases and diabetes, as well as the occurrence of lung cancer, and breast cancer. Furthermore, long-term exposure to high concentrations of the particulate matters has been proven to be associated with an increase in periodontal diseases and oral cancer.

Modern individuals are increasingly exposed to air pollution, which raises the risk of periodontal disease and oral cancer. In the advanced stages of periodontal disease and oral cancer, oral hygiene alone cannot fully alleviate symptoms, painkillers and anti-inflammatory medications can reduce pain and inflammation, but the structural changes to the gums remain irreversible. If more than 50% of the periodontium tissue is damaged, severe tooth loss may occur. Therefore, one of the current research objectives is to find ways to mitigate gum damage before gingivitis progresses to periodontitis and subsequently to oral cancer.

### SUMMARY

The present disclosure provides a new approach to ameliorating oral damage, opening up a new direction for the treatment of oral damage. The composition of the present disclosure is expected to be a medicament or composition for mitigating, repairing, ameliorating, or treating periodontal disease, periodontal abscess, oral submucous fibrosis, Leukoplakia, or oral cancer.

In one embodiment of the present disclosure, a use of mitochondria in manufacturing a composition for oral damage mitigation is provided.

In one embodiment of the present disclosure, a composition includes mitochondria and a biocompatible carrier.

In one embodiment of the present disclosure, a method for manufacturing a composition comprising mitochondria and extracellular vesicles includes: culturing cells with culture medium in a container; separating supernatant in the container from the cells adhering to the container after culturing; collecting extracellular vesicles from the supernatant; lysing the cells to isolate mitochondria inside the cells; and mixing the extracellular vesicles and the mitochondria to obtain the composition.

According to the embodiments of the present disclosure, the composition including mitochondria may reduce the damage caused by particulate matters to gingival fibroblasts, thereby reducing the death of gingival fibroblasts. Also, the composition including mitochondria may reduce the aging of gingival fibroblasts induced by particulate matters. Also, the composition including mitochondria may reduce the production of reactive oxygen species (ROS) in gingival fibroblasts induced by particulate matters, thereby reducing the further damage caused by the reactive oxygen species to gingival fibroblasts. In addition, the composition including mitochondria may reduce the damage caused by particulate matters to the mitochondria in gingival fibroblasts, thereby improving the mitochondrial function of gingival fibroblasts. Further, the composition including mitochondria and platelet-rich plasma-derived extracellular vesicles, the composition including mitochondria and stem cell-derived extracellular vesicles, and the composition including mitochondria and extracellular matrix exhibit synergistic effects in mitigating, repairing, ameliorating, or treating the damage to gingival fibroblasts, significantly reduce the aging or death of gingival fibroblasts caused by particulate matters, further reduce the production of reactive oxygen species and the associated damage, and further improve the mitochondrial function of gingival fibroblasts. Therefore, the composition of the embodiments of the present disclosure may achieve the purposes of mitigating, repairing, ameliorating, or treating oral damage, and may be expected to be a composition or medicament that is able to mitigate, repair, ameliorate or treat periodontal disease, periodontal abscess, oral submucous fibrosis, Leukoplakia or oral cancer while having both safety and effectiveness.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 shows the cell viability of the human gingival fibroblasts treated with PM, relative to the control group.

FIG. 2 shows the staining image for cell senescence of the human gingival fibroblasts treated with PM.

FIG. 3 shows the cell senescence level of the human gingival fibroblasts treated with PM.

FIG. 4 shows the ROS production of the human gingival fibroblasts treated with PM, relative to the control group.

FIG. 5 shows the ratio of JC-1 monomer/JC-1 aggregate (JC-1 ratio) of the mitochondria in the human gingival fibroblasts treated with PM.

FIG. 6 shows the ATP production of the mitochondria in the human gingival fibroblasts treated with PM.

FIG. 7 shows the cell viability of the human gingival fibroblasts treated with PM and then treated with the compositions of the examples and the comparative examples, relative to the control group.

FIG. 8 shows the cell viability of the human gingival fibroblasts treated with PM and then treated with the compositions of the examples and the comparative examples, relative to the control group.

FIG. 9 shows the cell viability of the human gingival fibroblasts treated with PM and then treated with the compositions of the examples and the comparative examples, relative to the control group.

FIG. 10 shows the cell senescence level of the human gingival fibroblasts treated with PM and then treated with the compositions of the examples and the comparative examples.

FIG. 11 shows the ROS production of the human gingival fibroblasts treated with PM and then treated with the compositions of the examples and the comparative examples, relative to the control group.

FIG. 12 shows the ROS production of the human gingival fibroblasts treated with PM and then treated with the compositions of the examples and the comparative examples, relative to the control group.

FIG. 13 shows the ROS production of the human gingival fibroblasts treated with PM and then treated with the compositions of the examples and the comparative examples, relative to the control group.

FIG. 14 shows the ratio of JC-1 monomer/JC-1 aggregate (JC-1 ratio) of the mitochondria in the human gingival fibroblasts treated with PM and then treated with the compositions of the examples and the comparative examples.

FIG. 15 shows the ratio of JC-1 monomer/JC-1 aggregate (JC-1 ratio) of the mitochondria in the human gingival fibroblasts treated with PM and then treated with the compositions of the examples and the comparative examples.

FIG. 16 shows the ratio of JC-1 monomer/JC-1 aggregate (JC-1 ratio) of the mitochondria in the human gingival fibroblasts treated with PM and then treated with the compositions of the examples and the comparative examples.

FIG. 17 shows the ATP production of the mitochondria in the human gingival fibroblasts treated with PM and then treated with the compositions of the examples and the comparative examples.

### DETAILED DESCRIPTION

In the following detailed description, for purposes of explanation, numerous specific details are set forth in order to provide a thorough understanding of the disclosed embodiments. According to the description, claims and the drawings disclosed in the specification, one skilled in the art may easily understand the concepts and features of the present invention. The following embodiments further illustrate various aspects of the present invention, but are not meant to limit the scope of the present invention.

In the present disclosure, features and conditions such as values, numbers, contents, amounts or concentrations presented as a range are merely for convenience and brevity. Therefore, a range should be interpreted as encompassing all possible subranges and individual numerals or values therein, including integers and non-integers. For example, a range of “1.0 to 4.0”, “1.0˜4.0” or “between 1.0 and 4.0” should be understood as explicitly disclosing all subranges such as 1.0 to 4.0, 1.0 to 3.0, 1.0 to 2.0, 2.0 to 4.0, 2.0 to 3.0, 3.0 to 4.0 and so on and encompassing the end points of the ranges, particularly, a subrange defined by numerals or values whose significant digits are represented by 0, 1, 2, 3, 4, 5, 6, 7, 8, 9 or 0 should be interpreted as encompassing all significant digits of numerals or values within the range defined by the end points, for example “1.00 to 2.00” should be interpreted as encompassing all individual values such as 1.00, 1.10, 1.20, 1.30, 1.40, 1.50, 1.60, 1.70, 1.80, 1.90 and so on.

Given the intended purposes and advantages of this disclosure are achieved, numerals have the precision of their significant digits. For example, 10.0 should be understood as covering a range of 9.50 to 10.49.

In one embodiment of the present disclosure, a composition includes mitochondria and a biocompatible carrier. The composition according to one embodiment of the present disclosure may act on gingival fibroblasts to mitigate, repair, ameliorate or treat oral damage. The oral damage may include oral diseases, and the oral diseases may include periodontal disease, periodontal abscess, oral submucous fibrosis, Leukoplakia or oral cancer.

The mitochondria may be taken from any cell having mitochondria, preferably taken from mammalian monocytes or stem cells, but not limited thereto. For example, the stem cells may be adipose-derived mesenchymal stem cells, embryonic stem cells, mesenchymal stem cells, hematopoietic stem cells, CD34+ stem cells, induced pluripotent stem cells or bone marrow stem cells. In some embodiments, the source of the mitochondria depends on a subject to which the composition is administered, and the mitochondria may be preferably taken from cells of the same species as the subject to which the composition is administered. For example, the mitochondria may be taken from human cells when the subject to which the composition is administered is human, while the mitochondria may be taken from dog cells when the subject to which the composition is administered is a dog. In some embodiment, the mitochondria may be taken from cells of species different from the subject to which the composition is administered, or the mitochondria may also be exogenous mitochondria obtained from in vitro preservation or in vitro culture. In some embodiments, the mitochondria may be used directly after being taken out, or may be used after in vitro preservation or in vitro culture.

The biocompatible carrier may maintain the mitochondrial activity, encapsulate the mitochondria, facilitate the mitochondria to enter into cells, or enhance mitochondrial targeting and specificity. The biocompatible carrier may include a pharmaceutically acceptable carrier. The pharmaceutically acceptable carrier may include a carrier used in any standard medical products or cosmetic products. The biocompatible carrier may be a semi-solid or liquid depending on the form of the composition. For example, the biocompatible carrier may include, but not limited to, water, physiological saline or buffer solution.

In the embodiment, the concentration of the mitochondria in the composition may be 40 μg/mL to 200 μg/mL. In another embodiment, the concentration of the mitochondria in the composition may be 40 μg/mL to 60 μg/mL. In other embodiment, the concentration of the mitochondria in the composition may be 60 μg/mL to 160 μg/mL. In other embodiment, the concentration of the mitochondria in the composition may be 160 μg/mL to 200 μg/mL. In other embodiment, the concentration of the mitochondria in the composition may be at least 60 μg/mL.

In the embodiment, the effective dose of the mitochondria in the composition may be 10 μg to 50 μg. In another embodiment, the effective dose of the mitochondria in the composition may be 10 μg to 15 μg. In other embodiment, the effective dose of the mitochondria in the composition may be 15 μg to 40 μg. In other embodiment, the effective dose of the mitochondria in the composition may be 40 μg to 50 μg. In other embodiment, the effective dose of the mitochondria in the composition may be at least 15 μg.

In the embodiment, the composition may be administered to the gingival fibroblasts in a manner of oral administration, injection, coating, topically applying, or dripping.

In another embodiment, the composition may further include extracellular matrix (ECM). The extracellular matrix is components synthesized and secreted by cells to the outside of the cells, and includes collagen, elastin, laminin, fibronectin, glycosaminoglycan (GAG), proteoglycan (PG), various growth factors and enzymes. The extracellular matrix functions to support and fix cells and provides a site for cell communication and regulation. The extracellular matrix plays a crucial role in cell growth and maintaining cell structures and functions. The extracellular matrix may be obtained commercially, through the decellularization process after cell culturing, or through enzyme treatment to preserve active components. The decellularization process may include rinsing the cells 3 times with PBS, removing the rinsing buffer, adding the cell lysis buffer and incubating at 37° C. for 15 minutes, removing the used cell lysis buffer, adding fresh lysis buffer and incubating at 37° C. for 40 to 60 minutes, removing the used cell lysis buffer, washing the cells 3 to 4 times with buffer solution, washing the cells 4 times with deionized water and then with PBS, and finally obtaining the extracellular matrix. The extracellular matrix is treated with proteolytic enzyme (such as trypsin), and the obtained supernatant is the soluble extracellular matrix (soluble ECM).

In the embodiment, the concentration of the extracellular matrix in the composition may be 5 mg/mL to 30 mg/mL. In another embodiment, the concentration of the extracellular matrix in the composition may be 5 mg/mL to 15 mg/mL. In other embodiment, the concentration of the extracellular matrix in the composition may be 15 mg/mL to 30 mg/mL. In other embodiment, the concentration of the extracellular matrix in the composition may be 15 mg/mL.

In the embodiment, the ratio of the extracellular matrix to the mitochondria in the composition may be 1 μg:2 μg to 1 μg:32 μg. In another embodiment, the ratio of the extracellular matrix to the mitochondria in the composition may be 1 μg:2 μg to 1 μg:5.3 μg. In other embodiment, the ratio of the extracellular matrix to the mitochondria in the composition may be 1 μg:15 μg to 1 μg:32 μg. In other embodiment, the ratio of the extracellular matrix to the mitochondria in the composition may be 1 μg:4 μg to 1 μg:10.67 μg.

In another embodiment, the composition may further include extracellular vesicles. The extracellular vesicles may be derived from platelet-rich plasma (PRP), stem cells monocytes, fibroblasts, neurons, smooth muscle cells, endothelial cells or epidermal cells. The stem cell may be mesenchymal stem cells (such as adipose-derived mesenchymal stem cells or umbilical cord-derived mesenchymal stem cells), hematopoietic stem cells, neural stem cells, embryonic stem cells, umbilical cord blood stem cells, amniotic stem cells, placental stem cells or induced pluripotent stem cells.

The manufacturing method of PRP-derived extracellular vesicles will be described in the following embodiments. The PRP-derived extracellular vesicles according to the embodiment of the present disclosure are vesicles with a lipid membrane structure, with a size between about 30 nm to 1000 nm. The vesicles encapsulate substances such as nucleic acids, peptides, proteins, and lipids. The PRP-derived extracellular vesicles express platelet-specific surface antigen CD41 as well as extracellular vesicle-specific surface antigens CD9, CD63 and Alix.

In the embodiment, the concentration of the PRP-derived extracellular vesicles in the composition may be 0.5 mg/mL to 2.5 mg/mL. In another embodiment, the concentration of the PRP-derived extracellular vesicles in the composition may be 0.5 mg/mL to 1 mg/mL. In other embodiment, the concentration of the PRP-derived extracellular vesicles in the composition may be 1.5 mg/mL to 2.5 mg/mL. In other embodiment, the concentration of the PRP-derived extracellular vesicles in the composition may be 1.25 mg/mL.

In the embodiment, the concentration of the PRP-derived extracellular vesicles in the composition may be 1% (v/v) to 5% (v/v). In another embodiment, the concentration of the PRP-derived extracellular vesicles in the composition may be 1% (v/v) to 2.5% (v/v). In other embodiment, the concentration of the PRP-derived extracellular vesicles in the composition may be 2.5% (v/v) to 5% (v/v). In other embodiment, the concentration of the PRP-derived extracellular vesicles in the composition may be 2.5% (v/v).

In the embodiment, the ratio of the PRP-derived extracellular vesicles to the mitochondria in the composition may be 1 mg:64 μg to 1 mg:320 μg. In another embodiment, the ratio of the PRP-derived extracellular vesicles to the mitochondria in the composition may be 1 mg:160 μg to 1 mg:320 μg. In other embodiment, the ratio of the PRP-derived extracellular vesicles to the mitochondria in the composition may be 1 mg:64 μg to 1 mg:106.6 μg. In other embodiment, the ratio of the PRP-derived extracellular vesicles to the mitochondria in the composition may be 1 mg:128 μg.

In the embodiment, the ratio of the PRP-derived extracellular vesicles to the mitochondria in the composition may be 1 μL:3.2 μg to 1 μL:16 μg. In another embodiment, the ratio of the PRP-derived extracellular vesicles to the mitochondria in the composition may be 1 μL:3.2 μg to 1 μL:6.4 μg. In other embodiment, the ratio of the PRP-derived extracellular vesicles to the mitochondria in the composition may be 1 μL:8 μg to 1 μL:16 μg. In other embodiment, the ratio of the PRP-derived extracellular vesicles to the mitochondria in the composition may be 1 μL:6.4 μg.

The manufacturing method of stem cell-derived extracellular vesicles will be described in the following embodiments. In the composition according to the embodiment of the present disclosure, the stem cell-derived extracellular vesicles and the mitochondria may be derived from the same stem cells. The stem cell-derived extracellular vesicles according to the embodiment of the present disclosure are vesicles with a lipid membrane structure, with a size between about 30 nm to 1000 nm. The vesicles encapsulate substances such as nucleic acids, peptides, proteins, and lipids. The stem cell-derived extracellular vesicles express surface antigens CD40, CD63, CD81, CD9, Alix, Hsp60, Hsp70 and Hsp90.

In the embodiment, the concentration of the stem cell-derived extracellular vesicles in the composition may be 1 μg/mL to 20 μg/mL. In another embodiment, the concentration of the stem cell-derived extracellular vesicles in the composition may be 1 μg/mL to 5 μg/mL. In other embodiment, the concentration of the stem cell-derived extracellular vesicles in the composition may be 15 μg/mL to 20 μg/mL. In other embodiment, the concentration of the stem cell-derived extracellular vesicles in the composition may be 10 μg/mL.

In the embodiment, the ratio of the stem cell-derived extracellular vesicles to the mitochondria may be 1 μg:8 μg to 1 μg:160 μg. In another embodiment, the ratio of the stem cell-derived extracellular vesicles to the mitochondria may be 1 μg:8 μg to 1 μg:10.6 μg. In other embodiment, the ratio of the stem cell-derived extracellular vesicles to the mitochondria may be 1 μg:32 μg to 1 μg:160 μg. In other embodiment, the ratio of the stem cell-derived extracellular vesicles to the mitochondria may be 1 μg:16 μg.

According to another embodiment of the present disclosure, a use of mitochondria in manufacturing a composition for mitigating, repairing, ameliorating or treating oral damage is provided. The composition may be the above-described composition including mitochondria. The oral damage may be damages of the gingival fibroblasts. The oral damage may include periodontal disease, periodontal abscess, oral submucous fibrosis, Leukoplakia or oral cancer. The composition may improve the mitochondrial membrane potential and the mitochondrial ATP production of the gingival fibroblasts, thereby improving the mitochondrial function of the gingival fibroblasts. The composition may reduce the death of the gingival fibroblasts, reduce the aging of the gingival fibroblasts, reduce reactive oxygen species produced by the gingival fibroblasts, or improve the mitochondrial function of the gingival fibroblasts, thereby mitigating, repairing, ameliorating or treating the oral damage.

According to another embodiment of the present disclosure, a method for manufacturing a composition for oral damage mitigation is provided. The method includes dispersing the mitochondria in the extracellular matrix to obtain the composition. In detail, the mitochondria and the extracellular matrix are well-mixed by, for example, shaking, using a stirring rod, pipetting, or magnetic stirring to make the mitochondria dispersed in the extracellular matrix to form the composition including the mitochondria and the extracellular matrix. The mixing time may be 5 to 60 minutes, such as 15 minutes, 30 minutes or 60 minutes, preferably no more than 60 minutes. The ratio of the extracellular matrix to the mitochondria may be 1 μg:2 μg to 1 μg:32 μg, preferably 1 μg:4 μg to 1 μg:10.67 μg. After the mitochondria and the extracellular matrix are mixed to form the composition, the composition may be subsequently administered on oral damage.

According to another embodiment of the present disclosure, a method for manufacturing a composition including mitochondria and extracellular vesicles includes culturing cells with medium in a container; separating supernatant in the container from the cells adhering to the container after culturing; collecting extracellular vesicles from the supernatant; lysing the cells to isolate mitochondria inside the cells; and mixing the extracellular vesicles and the mitochondria to obtain the composition. The method allows the simultaneous extraction of the mitochondria and the extracellular vesicles from the same cells.

In detail, the cell culture medium, culture container, and culture method may be selected based on the type of the cells to be cultured. After cell culture, the supernatant in the container may be separated from the cells adhering to the container by pouring or pipetting. The extracellular vesicles may be collected from the supernatant through tangential flow filtration (TFF). The cells may be collected by enzymatic digestion with trypsin and centrifugation, and the mitochondria in the cells may be isolated by lysing the cells through physical grinding or chemical lysis. The obtained extracellular vesicles and the obtained mitochondria are mixed to form the composition. The mixing ratio of the extracellular vesicles and the mitochondria may be 1 μg:8 μg to 1 μg:160 μg, such as 1 μg:16 μg. The steps of collecting the extracellular vesicles and isolating the mitochondria may be performed simultaneously or sequentially, and the order is not limited. The cells used in the method may be stem cells, such as mesenchymal stem cells (such as adipose-derived mesenchymal stem cells or umbilical cord-derived mesenchymal stem cells), hematopoietic stem cells, neural stem cells, embryonic stem cells, umbilical cord blood stem cells, amniotic stem cells, placental stem cells or induced pluripotent stem cells.

The materials used in the experiments are described as follows.

The mitochondria used in the embodiment of the present disclosure are taken from human adipose-derived mesenchymal stem cells (ADSCs), and the adipose-derived mesenchymal stem cells express surface makers CD73, CD90 and CD105, and no surface markers CD34 and CD45. The culture medium for the stem cells includes Keratinocyte SFM 1× solution (Gibco), bovine pituitary extract (BPE, Gibco), 10% (v/v) FBS (HyClone). First, ADSCs are cultured in a Petri dish to 1.5×10⁸ cells and then washed with Dulbecco's phosphate-buffered saline (DPBS). Next, DPBS is removed, trypsin for dissociating adherent cells from the Petri dish surfaces is added and reacted at 37° C. for 3 minutes, and the reaction is stopped by adding the culture medium for the stem cells. Next, ADSCs are washed down from the Petri dish, dispersed, and centrifuged at 600 g for 10 minutes, and the supernatant is removed. Next, the remained ADSCs and 80 mL of IBC-1 buffer (225 mM mannitol, 75 mM sucrose, 0.1 mM EDTA, and 30 mM Tris-HCl pH 7.4) are added to a homogenizer, and ADSCs are ground on ice by the homogenizer. Next, the ground ADSCs are centrifuged at 600 g and 4° C. for 5 minutes, and the supernatant is collected. Next, the supernatant is centrifuged at 10000 g and 4° C. for 10 minutes, and then the supernatant is removed. The mitochondria are extracted by using Mitochondria Isolation Kit, human (purchased from Miltenyi Biotec, Germany). Magnetic microbead antibody Anti-TOM22 is added to the resulting extract and reacted on ice for 1 hour, and then the mitochondria are purified by magnetic separation. The protein concentration is measured for the purified mitochondria and defined as the weight of the mitochondria.

The extracellular matrix (ECM) used in the embodiments of the present disclosure is purchased from Sigma (MaxGel™ ECM, E0282). The extracellular matrix includes human extracellular matrix components, including collagen, laminin, fibronectin, tenascins, elastin, proteoglycan and glycosaminoglycan. In the below experiments, the mitochondria and the extracellular matrix are mixed, and the mitochondria are dispersed in the extracellular matrix to form the composition including the mitochondria and the extracellular matrix. After mixing for 15 minutes, the composition is administered to the experimental cells.

The PRP-derived extracellular vesicles used in the embodiments of the present disclosure may be manufactured as follows. Whole blood is collected from the median cubital vein using a 19 G butterfly needle, and the first 5 mL of the collected blood is discarded to collect about 20 mL of blood. The collected blood is placed in a 50 mL centrifuge tube containing 3.2% (v/v) of trisodium citrate. The blood is centrifuged at 2500 g for 15 minutes to collect about 5 to 6 mL of supernatant, which is platelet-rich plasma (PRP). 5 to 6 mL of the obtained PRP is mixed with phosphate-buffered saline (PBS) (without calcium and magnesium ions) at a ratio of 1:1, centrifuged at 10000 g and 4° C. for 120 minutes, and the supernatant is removed to obtain 30 to 70 mg of the PRP-derived extracellular vesicles. The obtained PRP-derived extracellular vesicles are resuspended with 1 mL of PBS to obtain about 50.05±17.66 mg/mL of the extracellular vesicles of PRP, hereinafter referred to as PRP-EVs. The PRP-derived extracellular vesicles express platelet-specific surface antigen CD41 as well as extracellular vesicle-specific surface antigens CD9, CD63 and Alix.

The stem cell-derived extracellular vesicles (MSC-derived extracellular vesicles) used in the embodiment of the present disclosure may be manufactured as follows. In the composition including the mitochondria and the stem cell-derived extracellular vesicles according to the embodiment of the present disclosure, the mitochondria and the extracellular vesicles are derived from the same stem cells. The stem cells used in the embodiment are adipose-derived mesenchymal stem cells. The culture medium for the stem cells includes Keratinocyte SFM 1× solution (Gibco), EGF (Gibco), bovine pituitary extract (BPE, Gibco), N-acetyl-L-cysteine (Sigma), L-ascorbic acid 2-phosphate (magnesium salt hydrate) (Sigma), 10% (v/v) FBS (HyClone). First, the stem cells are cultured until the culture dish is 80% full, and then the medium is replaced with fresh culture medium to culture for 24 hours. Then, the culture medium is removed, and the cells are rinsed with PBS. Then, the rinsed PBS is removed, and fresh culture medium is added to culture for 48 hours. After culture, the supernatant in the culture dish and the stem cells adhering to the culture dish are separated by a pipette.

175 mL of the supernatant is filtered through a 0.22 m filter. The extracellular vesicles are purified and concentrated from the supernatant using a tangential flow filtration (TFF) system (100 kDa mPES filter membrane, D02-E100-05-N) to obtain 35 mL of purified supernatant. The substances contained in the purified supernatant are defined as extracellular vesicles. The stem cells adhering to the culture dish are collected by enzymatic digestion with trypsin, and ground to extract and purify the mitochondria, as described in the method of extracting mitochondria. The method allows the simultaneous extraction of the mitochondria and the extracellular vesicles from the same stem cells.

In the following experiments, the human gingival fibroblasts (HGFs) are used to study oral damages. The culture medium of the human gingival fibroblasts may include DMEM (Dulbecco's Modified Eagle Medium), 4.5 g/L D-glucose, 110 mg/L sodium pyruvate, 584 mg/L glutamine, 3.7 g/L sodium bicarbonate, and 10% (v/v) fetal bovine serum (FBS). The human gingival fibroblasts are cultured at a density of 3000 to 6000 cells/cm² at 37° C. in the above culture medium for subculturing. The human gingival fibroblasts are cultured until the culture dish is 90% full, then the culture medium is removed, and the cells are rinsed with phosphate buffered saline (PBS). Then, PBS is removed, 0.25% of Trypsin is added to the culture dish and incubated at 37° C. for 5 minutes, and then fresh culture medium is added to stop the trypsin reaction. Then, the cells are centrifuged at 300 g for 5 minutes to remove the supernatant, and then fresh culture medium is added thereto to count the cells, and the cell subculture will be performed according to experimental requirements.

In the following experiments, urban particulate matters (purchased from Merck, NIST1648A) are used as the damaging agent for the human gingival fibroblasts. Hereinafter, the urban particulate matters will be referred to as particulate matters or PM. 0.01 g PM is placed in Eppendorf and added with 1 mL PBS. The Eppendorf is sealed with Parafilm and shaken for 1 hour using an ultrasonic water bath shaker. After shaking, the solution is used as the stock solution (with a concentration of 10 μg/μL) and stored in a refrigerator of 4° C. for subsequent experiments.

In the following experiments, CCK-8 kit (purchased from Dojindo, CK04) is used to analyze cell viability. WST-8 is a main reagent in the CCK-8 kit, and WST-8 has low cytotoxicity, high sensitivity, strong water solubility, and is easy to store. WST-8 reacts with dehydrogenase in living cells and is reduced from pink to orange (Formazan dye). The amount of the produced formazan is proportional to the number of living cells. Therefore, the cell viability may be analyzed by measuring the absorbance (OD 450 nm) resulting from formazan using a spectrophotometer in a cytotoxicity test or a cell proliferation test.

In the following experiments, SA-β-gal kit (Senescence β-Galactosidase Staining Kit #9860, purchased from Cell Signaling technology) is used to evaluate the cell senescence level. In the aged cells, senescence-associated beta-galactosidase (SA-β-gal) is overexpressed, and thus SA-β-gal may be a biomarker of cellular senescence level. Therefore, the cell senescence may be observed through SA-β-gal staining.

In the following experiments, CM-H₂DCFDA (purchased from Invitrogen, C6827) is used to analyze reactive oxygen species (ROS) in cells. CM-H₂DCFDA may permeate the cell membrane and reacts with ROS inside the cells to form high-fluorescent products, and thus it is often used as an indicator for ROS.

In the following experiments, JC-1 dye (Invitrogen T3168, purchased from Fisher scientific) is used to analyze the mitochondrial membrane potential of cells. When the mitochondrial function of the cells is normal, the mitochondria are polarized, and the mitochondrial membrane potential is negatively charged. At this time, the positively charged JC-1 dye is accumulated on the mitochondrial membrane to form JC-1 aggregate and generate red fluorescence. When the mitochondrial function is damaged, the mitochondria are depolarized, and the mitochondrial membrane potential collapses. At this time, JC-1 dye does not form aggregate, and JC-1 monomers are distributed in the cells and generate green fluorescence. Therefore, a ratio of JC-1 monomer/JC-1 aggregate (hereinafter referred to as JC-1 ratio) may be obtained by fluorescent measurement and may be used as an indicator for evaluating the mitochondrial function. When JC-1 monomer/JC-1 aggregate is high, the mitochondrial membrane potential is poor, which indicates the mitochondrial function of the cells is poor.

In the following experiments, ATP assay kit (purchased from BioVision, K354-100) is used to analyze the amount of ATP produced by the mitochondria in the cells. One of the important functions of mitochondria is to generate ATP through the electron transport chain for cellular use. If the mitochondria are damaged, their ability to generate ATP will also be affected. Therefore, ATP production may be measured to indicate the mitochondrial ATP-production capacity and may be used as an indicator for evaluating mitochondrial function.

Unless otherwise stated, the experimental values are presented as mean±standard deviation and are statistically analyzed by ANOVA test and Tukey post hoc test.

### Experiment 1: Cytotoxicity of Particulate Matters (PM) to Human Gingival Fibroblasts

The human gingival fibroblasts are cultured at a density of 40000 cells per well in 0.5 mL of the culture medium in a 24-well plate, in which the bottom area of each well is 1.8 cm², for 24 hours. Then, after the cells are cultured until the well is 80% full, the culture medium is removed, and the cells are rinsed with 0.5 mL PBS per well. Then, the rinsed PBS is removed, and a fresh DMEM with 1% FBS (250 μL/well) is added. Then, PM is added at a concentration of 0, 25, 50, or 100 μg/cm² in the well. After the cells are cultured with PM at 37° C. and 5% CO₂ for 24 hours, the cell viability is analyzed by using a CCK-8 kit.

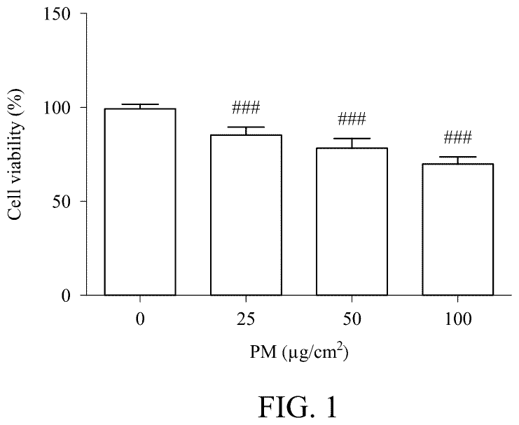

The experimental results are shown in Table 1 and FIG. 1. FIG. 1 shows the cell viability of the human gingival fibroblasts treated with PM, relative to the control group. In FIG. 1, the control group is the cells without PM (PM is 0 μg/cm²), and the symbol “#” represents a statistically significant difference (### indicates P<0.001) relative to the control group. From the experimental results, PM causes damage to the human gingival fibroblasts. In addition, the extent of the damage becomes more severe as the concentration of PM increases.

TABLE 1

| PM (μg/cm²) | Cell viability (%) |
|---|---|
| 0 | 100 ± 2.2 |
| 25 | 83.4 ± 1.3 |
| 50 | 73.6 ± 1.4 |
| 100 | 66.9 ± 0.7 |

### Experiment 2: Senescence of the Human Gingival Fibroblasts Induced by PM

The procedure in this experiment is generally the same as that in Experiment 1, and only the differences are described below. PM is added at a concentration of 0, 25, 50, or 100 μg/cm² in the well. After the cells are cultured with PM at 37° C. and 5% CO₂ for 24 hours, the senescence level of the cells is evaluated by using a SA-β-gal kit.

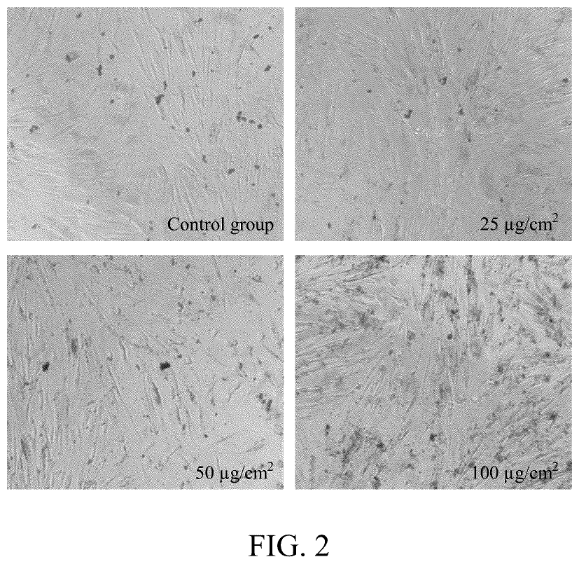

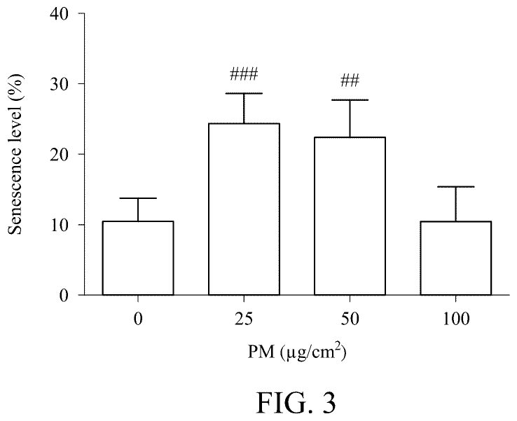

The experimental results are shown in Table 2, FIGS. 2 and 3. FIG. 2 shows the staining image for cell senescence of the human gingival fibroblasts treated with PM. FIG. 3 shows the cell senescence level of the human gingival fibroblasts treated with PM. In FIG. 2 and FIG. 3, the control group is the cells without PM (PM is 0 μg/cm²), and the symbol “#” represents a statistically significant difference (## indicates P<0.01, and ### indicates P<0.001) relative to the control group. From the experimental results, PM induces aging of the human gingival fibroblasts. In addition, the concentration of PM increases, but the senescence level decreases. It is hypothesized that the reason may be that a higher concentration of PM (50 μg/cm² and 100 μg/cm²) causes cell death, resulting in a decrease in the stained cells, thereby showing a lower senescence level.

TABLE 2

| PM (μg/cm²) | Senescence level (%) |
|---|---|
| 0 | 10.7 ± 3.0 |
| 25 | 24.7 ± 4.0 |
| 50 | 22.6 ± 5.0 |
| 100 | 10.7 ± 4.7 |

### Experiment 3: Reactive Oxygen Species (ROS) Production of the Human Gingival Fibroblasts Induced by PM

The procedure in this experiment is generally the same as that in Experiment 1, and only the differences are described below. The human gingival fibroblasts are cultured for 24 hours, the culture medium is removed, and the cells are rinsed with PBS. Then, the rinsed PBS is removed, and a fresh DMEM with 1% FBS and 10 μM CM-H₂DCFDA (250 μL/well) is added to react in the dark at 37° C. for 45 minutes. After reaction, the supernatant in the well is removed, and the cells are rinsed with 0.5 mL PBS per well. Then, the rinsed PBS is removed, and a fresh DMEM with 1% FBS (250 μL/well) is added. Then, PM is added at a concentration of 0, 10, 25, or 50 μg/cm² in the well. The cells are cultured with PM at 37° C. and 5% CO₂ in the dark for 24 hours. After cell culture, the supernatant in the well is removed, and the cells are rinsed with 0.5 mL PBS per well. Then, the rinsed PBS is removed, and 250 μL per well of RIPA Lysis and Extraction Buffer (purchased from Thermo Scientific, 89900) is added to lysis the cells. The obtained solution is collected in a 1.5 mL tube and centrifuged at 300 g for 1 minute. 200 μL of the supernatant is loaded in a 96-well black plate, and the fluorescence is measured at OD485 (excitation) and OD530 (emission) to analyze the amount of ROS.

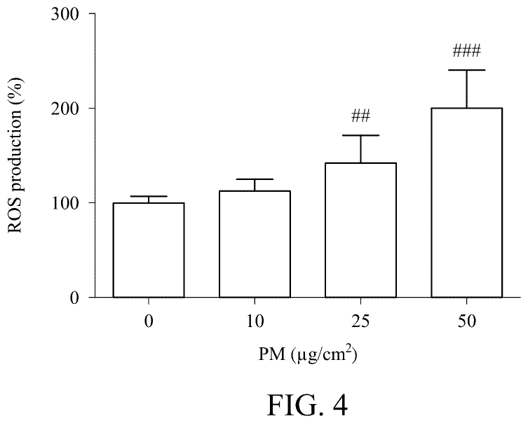

The experimental results are shown in Table 3 and FIG. 4. FIG. 4 shows the ROS production of the human gingival fibroblasts treated with PM, relative to the control group. In FIG. 4, the control group is the cells without PM (PM is 0 μg/cm²), and the symbol “#” represents a statistically significant difference (## indicates P<0.01, and ### indicates P<0.001) relative to the control group. From the experimental results, PM induces the human gingival fibroblasts to produce ROS. In addition, the produced ROS increases as the concentration of PM increases. The production and increase of ROS further cause oxidative damage to the human gingival fibroblasts.

TABLE 3

| PM (μg/cm²) | ROS production (%) |
|---|---|
| 0 | 100 ± 6.9 |
| 10 | 112.9 ± 11.8 |
| 25 | 141.7 ± 29.4 |
| 50 | 199.7 ± 40.4 |

### Experiment 4: Damage to the Mitochondria in the Human Gingival Fibroblasts Caused by PM—Membrane Potential Analysis

The procedure in this experiment is generally the same as that in Experiment 1, and only the differences are described below. PM is added at a concentration of 0, 10, 25, or 50 μg/cm² in the well. After the cells are cultured with PM at 37° C. and 5% CO₂ for 24 hours, the supernatant in the well is removed, and the cells are rinsed with 0.5 mL PBS per well. Then, the rinsed PBS is removed, and a fresh DMEM with 1% FBS and 5 μM JC-1 (250 μL/well) is added to react at 37° C. for 10 minutes. After reaction, the supernatant in the well is removed, and the cells are rinsed with 0.5 mL PBS per well twice. Then, a fresh DMEM with 1% FBS (250 μL/well) is added. The fluorescence of JC-1 aggregate is measured at OD520 (excitation) and OD590 (emission), and the fluorescence of JC-1 monomer is measured at OD490 (excitation) and OD530 (emission), thereby evaluating the mitochondrial membrane potential of the human gingival fibroblasts.

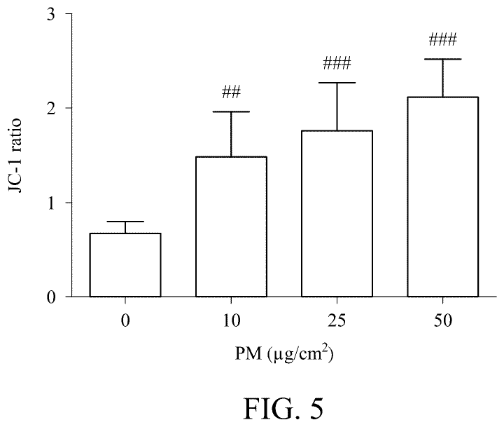

The experimental results are shown in Table 4 and FIG. 5. FIG. 5 shows the ratio of JC-1 monomer/JC-1 aggregate (JC-1 ratio) of the mitochondria in the human gingival fibroblasts treated with PM. In FIG. 5, the control group is the cells without PM (PM is 0 μg/cm²), and the symbol “#” represents a statistically significant difference (## indicates P<0.01, and ### indicates P<0.001) relative to the control group. From the experimental results, PM increases the ratio of JC-1 monomer/JC-1 aggregate (JC-1 ratio) of the mitochondria in the human gingival fibroblasts, which indicates that the mitochondrial membrane is damaged, and the mitochondrial function is damaged. In addition, the extent of the damage to the mitochondria becomes more severe as the concentration of PM increases.

TABLE 4

| PM (μg/cm²) | JC-1 ratio |
|---|---|
| 0 | 0.67 ± 0.12 |
| 10 | 1.48 ± 0.48 |
| 25 | 1.76 ± 0.51 |
| 50 | 2.11 ± 0.41 |

### Experiment 5: Damage to the Mitochondria in the Human Gingival Fibroblasts Caused by PM—ATP Production

The procedure in this experiment is generally the same as that in Experiment 1, and only the differences are described below. The human gingival fibroblasts are cultured at a density of 3.5×10⁵ cells in 10 mL of DMEM with 10% FBS in a 10 cm dish, in which the bottom area of the dish is 60.8 cm², for 24 hours. Then, after the cells are cultured until the dish is 80% full, the culture medium is removed, and the cells are rinsed with 10 mL PBS per dish. Then, the rinsed PBS is removed, and a fresh DMEM with 1% FBS (5 mL/dish) is added. Then, PM is added at a concentration of 0, 10, 25, or 50 μg/cm² in the dish. After the cells are cultured with PM for 24 hours, the ATP production of the mitochondria in the cells is analyzed by using an ATP assay kit.

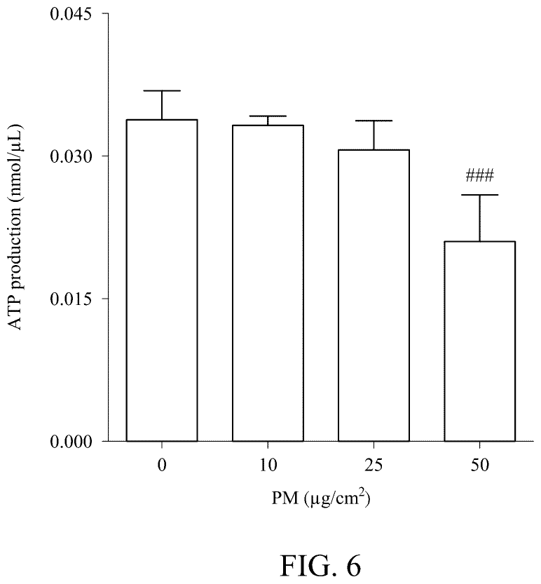

The experimental results are shown in Table 5 and FIG. 6. FIG. 6 shows the ATP production of the mitochondria in the human gingival fibroblasts treated with PM. In FIG. 6, the control group is the cells without PM (PM is 0 μg/cm²), and the symbol “#” represents a statistically significant difference (### indicates P<0.001) relative to the control group. From the experimental results, PM decreases the ATP production of the mitochondria in the human gingival fibroblasts, which indicates that the mitochondrial ATP-production capacity is damaged, and the mitochondrial function is damaged. In addition, the extent of the damage to the mitochondria becomes more severe as the concentration of PM increases.

TABLE 5

| PM (μg/cm²) | ATP production (nmol/μL) |
|---|---|
| 0 | 0.034 ± 0.003 |
| 10 | 0.033 ± 0.001 |
| 25 | 0.031 ± 0.003 |
| 50 | 0.021 ± 0.005 |

### Experiment 6: Mitochondria Reducing Cell Death of the Human Gingival Fibroblasts Caused by PM

The procedure in this experiment is generally the same as that in Experiment 1, and only the differences are described below. PM is added at a concentration of 0 or 50 μg/cm² in the well. After the cells are cultured with PM at 37° C. and 5% CO₂ for 6 hours, the cells are rinsed with 0.5 mL PBS per well. Then, the rinsed PBS is removed, and a fresh DMEM with 1% FBS (250 μL/well) and the composition of each of the examples and comparative examples are added to culture at 37° C. and 5% CO₂ for 20 hours. After cell culture, the cell viability is analyzed by using a CCK-8 kit.

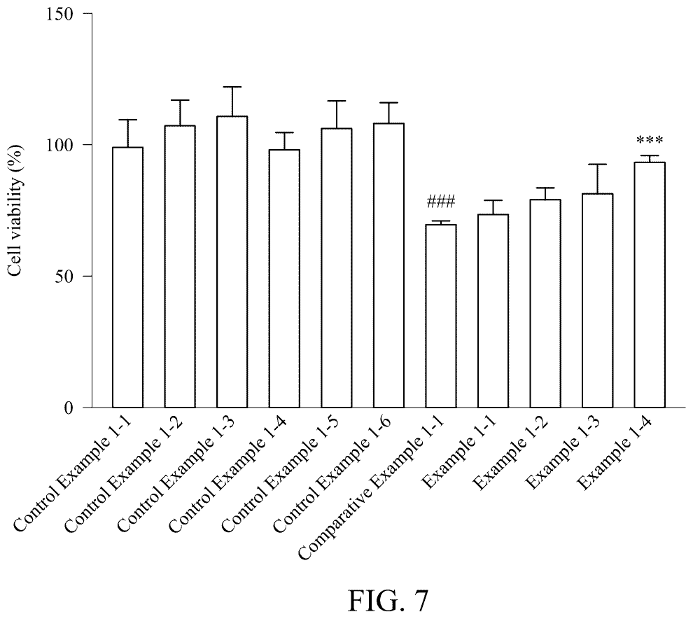

The experimental results for the composition including the extracellular matrix (ECM) and the mitochondria are shown in Table 6 and FIG. 7. FIG. 7 shows the cell viability of the human gingival fibroblasts treated with PM and then treated with the compositions of the examples and the comparative examples, relative to the control group. In FIG. 7, the control group is the cells without PM, mitochondria, and ECM (Control Example 1-1), the symbol “#” represents a statistically significant difference (### indicates P<0.001) relative to the control group (Control Example 1-1), and the symbol “*” represents a statistically significant difference (*** indicates P<0.001) relative to the comparative example (Comparative Example 1-1). From Control Examples 1-1 to 1-6, in the case that the cells are not damaged, the addition of the mitochondria or ECM alone does not decrease the cell viability and can even slightly increase the cell viability, which indicates that the mitochondria and ECM have no cytotoxicity to the human gingival fibroblasts, and it can be even said that the composition including the mitochondria or the composition including the mitochondria and ECM promotes the growth of the human gingival fibroblasts. In addition, from the examples and the comparative examples, in the case that the cells are damaged, the addition of the mitochondria can increase the cell viability (Examples 1-1 and 1-2), which indicates that the addition of the mitochondria contributes to mitigating, repairing, ameliorating, or treating the damage to the human gingival fibroblasts caused by PM, further reducing the death of the human gingival fibroblasts caused by PM. Further, from the examples and the comparative examples, in the case that the cells are damaged, the addition of the composition including the mitochondria and ECM can further increase the cell viability (Examples 1-3 and 1-4) and have a statistically significant difference, which indicates the addition of the composition including the mitochondria and ECM exhibits a synergistic effect in mitigating, repairing, ameliorating, or treating the damage to the human gingival fibroblasts, and can significantly reduce the death of the human gingival fibroblasts caused by PM.

TABLE 6

| | PM | Mitochondria | | ECM | Cell viability |
|---|---|---|---|---|---|
| Group | μg/cm² | μg | μg/mL | μg/mL | % |
| Control | — | — | — | — | 100 ± 9.7 |
| Example 1-1 | | | | | |
| Control | | 15 | 60 | — | 108.1 ± 8.9 |
| Example 1-2 | | | | | |
| Control | | 40 | 160 | — | 111.8 ± 10.3 |
| Example 1-3 | | | | | |
| Control | | — | — | 15 | 99.2 ± 5.6 |
| Example 1-4 | | | | | |
| Control | | 15 | 60 | 15 | 107 ± 9.6 |
| Example 1-5 | | | | | |
| Control | | 40 | 160 | 15 | 111.7 ± 8.9 |
| Example 1-6 | | | | | |
| Comparative | 50 | — | — | — | 70.6 ± 0.5 |
| Example 1-1 | | | | | |
| Example 1-1 | | 15 | 60 | — | 74.3 ± 4.7 |
| Example 1-2 | | 40 | 160 | — | 80.2 ± 3.6 |
| Example 1-3 | | 15 | 60 | 15 | 82.4 ± 10.2 |
| Example 1-4 | | 40 | 160 | 15 | 94.4 ± 1.5 |

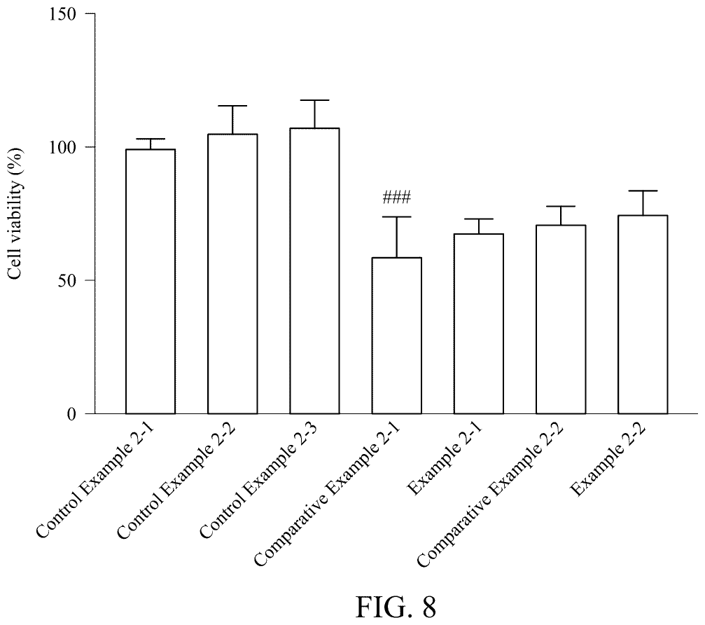

The experimental results for the composition including the PRP-derived extracellular vesicles (PRP-EVs) and the mitochondria are shown in Table 7 and FIG. 8. FIG. 8 shows the cell viability of the human gingival fibroblasts treated with PM and then treated with the compositions of the examples and the comparative examples, relative to the control group. In FIG. 8, the control group is the cells without PM, mitochondria, and PRP-EVs (Control Example 2-1), the symbol “#” represents a statistically significant difference (### indicates P<0.001) relative to the control group (Control Example 2-1). From Control Examples 2-1 to 2-3, in the case that the cells are not damaged, the addition of the mitochondria or PRP-EVs alone does not decrease the cell viability and can even slightly increase the cell viability, which indicates that the mitochondria and PRP-EVs have no cytotoxicity to the human gingival fibroblasts, and it can be even said that the composition including the mitochondria promotes the growth of the human gingival fibroblasts. In addition, from the examples and the comparative examples, in the case that the cells are damaged, the addition of the mitochondria can increase the cell viability (Example 2-1), which indicates that the addition of the mitochondria contributes to mitigating, repairing, ameliorating, or treating the damage to the human gingival fibroblasts caused by PM, further reducing the death of the human gingival fibroblasts caused by PM. Further, from the examples and the comparative examples, in the case that the cells are damaged, the addition of the composition including the mitochondria and PRP-EVs can further increase the cell viability (Example 2-2), which indicates the addition of the composition including the mitochondria and PRP-EVs exhibits a synergistic effect in mitigating, repairing, ameliorating, or treating the damage to the human gingival fibroblasts, and can significantly reduce the death of the human gingival fibroblasts caused by PM.

TABLE 7

| | PM | Mitochondria | | PRP-EVs | Cell viability |
|---|---|---|---|---|---|
| Group | μg/cm² | μg | μg/mL | v/v % | % |
| Control | — | — | — | — | 100 ± 3.1 |
| Example 2-1 | | | | | |
| Control | | 40 | 160 | — | 105.7 ± 9.8 |
| Example 2-2 | | | | | |
| Control | | — | — | 2.5 | 108.0 ± 9.6 |
| Example 2-3 | | | | | |
| Comparative | 50 | — | — | — | 59.4 ± 14.5 |
| Example 2-1 | | | | | |
| Example 2-1 | | 40 | 160 | — | 68.2 ± 4.8 |
| Comparative | | — | — | 2.5 | 69.1 ± 8.2 |
| Example 2-2 | | | | | |
| Example 2-2 | | 40 | 160 | 2.5 | 75.2 ± 8.3 |

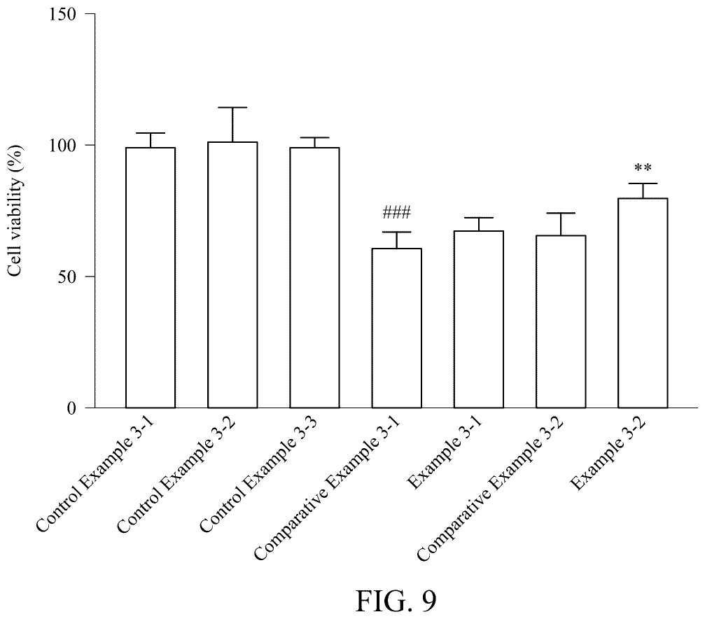

The experimental results for the composition including the MSC-derived extracellular vesicles (MSC-EVs) and the mitochondria are shown in Table 8 and FIG. 9. FIG. 9 shows the cell viability of the human gingival fibroblasts treated with PM and then treated with the compositions of the examples and the comparative examples, relative to the control group. In FIG. 9, the control group is the cells without PM, mitochondria, and MSC-EVs (Control Example 3-1), the symbol “#” represents a statistically significant difference (### indicates P<0.001) relative to the control group (Control Example 3-1), and the symbol “*” represents a statistically significant difference (** indicates P<0.01) relative to the comparative example (Comparative Example 3-1). From Control Examples 3-1 to 3-3, in the case that the cells are not damaged, the addition of the mitochondria or MSC-EVs alone does not decrease the cell viability, which indicates that the mitochondria and MSC-EVs have no cytotoxicity to the human gingival fibroblasts. In addition, from the examples and the comparative examples, in the case that the cells are damaged, the addition of the mitochondria can increase the cell viability (Example 3-1), which indicates that the addition of the mitochondria contributes to mitigating, repairing, ameliorating, or treating the damage to the human gingival fibroblasts caused by PM, further reducing the death of the human gingival fibroblasts caused by PM. Further, from the examples and the comparative examples, in the case that the cells are damaged, the addition of the composition including the mitochondria and MSC-EVs can further increase the cell viability (Example 3-2), which indicates the addition of the composition including the mitochondria and MSC-EVs exhibits a synergistic effect in mitigating, repairing, ameliorating, or treating the damage to the human gingival fibroblasts, and can significantly reduce the death of the human gingival fibroblasts caused by PM.

TABLE 8

| | PM | Mitochondria | | MSC-EVs | Cell viability |
|---|---|---|---|---|---|
| Group | μg/cm² | μg | μg/mL | μg/mL | % |
| Control | — | — | — | — | 100 ± 4.6 |
| Example 3-1 | | | | | |
| Control | | 40 | 160 | — | 102.1 ± 12.2 |
| Example 3-2 | | | | | |
| Control | | — | — | 10 | 99.9 ± 2.9 |
| Example 3-3 | | | | | |
| Comparative | 50 | — | — | — | 61.6 ± 5.3 |
| Example 3-1 | | | | | |
| Example 3-1 | | 40 | 160 | — | 68.3 ± 4.2 |
| Comparative | | — | — | 10 | 66.4 ± 7.9 |
| Example 3-2 | | | | | |
| Example 3-2 | | 40 | 160 | 10 | 80.7 ± 4.8 |

### Experiment 7: Mitochondria Reducing Aging of the Human Gingival Fibroblasts Caused by PM

The procedure in this experiment is generally the same as that in Experiment 1, and only the differences are described below. PM is added at a concentration of 0 or 25 μg/cm² in the well. After the cells are cultured with PM at 37° C. and 50% CO₂ for 6 hours, the cells are rinsed with 0.5 mL PBS per well. Then, the rinsed PBS is removed, and a fresh DMVEM with 10% FBS (250 μL/well) and the composition of each of the examples and comparative examples are added to culture at 37° C. and 50% CO₂ for 20 hours. After cell culture, the cell senescence level is evaluated by using a SA-β-gal kit.

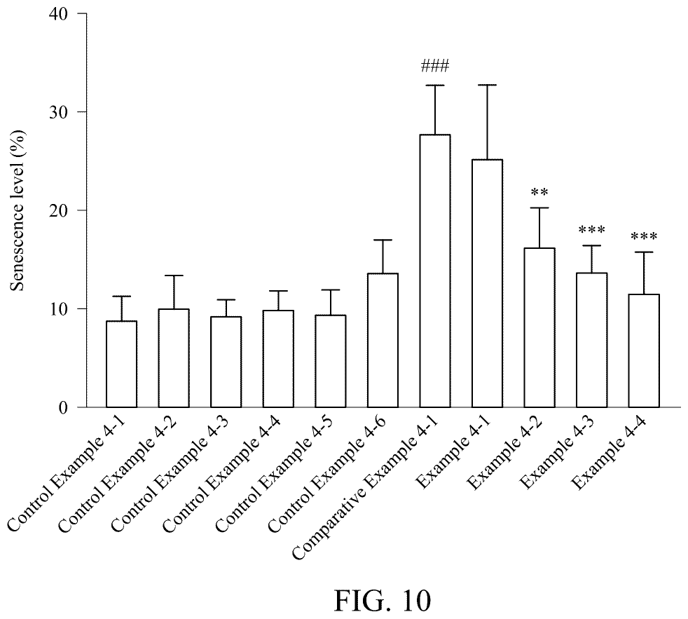

The experimental results are shown in Table 9 and FIG. 10. FIG. 10 shows the cell senescence level of the human gingival fibroblasts treated with PM and then treated with the compositions of the examples and the comparative examples. In FIG. 10, the control group is the cells without PM, mitochondria, and ECM (Control Example 4-1), the symbol “#” represents a statistically significant difference (### indicates P<0.001) relative to the control group (Control Example 4-1), and the symbol “*” represents a statistically significant difference (** indicates P<0.01, and *** indicates P<0.001) relative to the comparative example (Comparative Example 4-1). From Control Examples 4-1 to 4-6, in the case that the cells are not damaged, the addition of the mitochondria or ECM alone does not induce the cell senescence. In addition, from the examples and the comparative examples, in the case that the cells are damaged, the addition of the mitochondria can decrease the cell senescence level (Examples 4-1 and 4-2), which indicates that the addition of the mitochondria contributes to mitigating, repairing, ameliorating, or treating the damage to the human gingival fibroblasts caused by PM, further reducing the aging of the human gingival fibroblasts caused by PM. Further, from the examples and the comparative examples, in the case that the cells are damaged, the addition of the composition including the mitochondria and ECM can further decrease the cell senescence level (Examples 4-3 and 4-4) and have a statistically significant difference, which indicates the addition of the composition including the mitochondria and ECM exhibits a synergistic effect in mitigating, repairing, ameliorating, or treating the damage to the human gingival fibroblasts, and can significantly reduce the aging of the human gingival fibroblasts caused by PM.

TABLE 9

| | PM | Mitochondria | | ECM | Senescence level |
|---|---|---|---|---|---|
| Group | μg/cm² | μg | μg/mL | μg/mL | % |
| Control | — | — | — | — | 9.02 ± 2.29 |
| Example 4-1 | | | | | |
| Control | | 15 | 60 | — | 10.22 ± 3.18 |
| Example 4-2 | | | | | |
| Control | | 40 | 160 | — | 9.43 ± 1.52 |
| Example 4-3 | | | | | |
| Control | | — | — | 15 | 10.07 ± 1.75 |
| Example 4-4 | | | | | |
| Control | | 15 | 60 | 15 | 9.62 ± 2.33 |
| Example 4-5 | | | | | |
| Control | | 40 | 160 | 15 | 13.83 ± 3.21 |
| Example 4-6 | | | | | |
| Comparative | 25 | — | — | — | 27.92 ± 4.80 |
| Example 4-1 | | | | | |
| Example 4-1 | | 15 | 60 | — | 25.40 ± 7.32 |
| Example 4-2 | | 40 | 160 | — | 16.47 ± 3.83 |
| Example 4-3 | | 15 | 60 | 15 | 13.92 ± 2.54 |
| Example 4-4 | | 40 | 160 | 15 | 11.75 ± 4.05 |

### Experiment 8: Mitochondria Reducing ROS Production of the Human Gingival Fibroblasts Induced by PM

The procedure in this experiment is generally the same as that in Experiment 1, and only the differences are described below. The human gingival fibroblasts are cultured for 24 hours, the culture medium is removed, and the cells are rinsed with PBS. Then, the rinsed PBS is removed, and a fresh DMEM with 1% FBS and 10 μM CM-H₂DCFDA (250 μL/well) is added to react in the dark at 37° C. for 45 minutes. After reaction, the supernatant in the well is removed, and the cells are rinsed with 0.5 mL PBS per well. Then, the rinsed PBS is removed, and a fresh DMEM with 1% FBS (250 μL/well) is added. Then, PM is added at a concentration of 0 or 50 μg/cm² in the well. After the cells are cultured with PM at 37° C. and 5% CO₂ for 6 hours, the cells are rinsed with 0.5 mL PBS per well. Then, the rinsed PBS is removed, and a fresh DMEM with 1% FBS (250 μL/well) and the composition of each of the examples and comparative examples are added to culture at 37° C. and 5% CO₂ for 20 hours. After cell culture, the supernatant in the well is removed, and the cells are rinsed with 0.5 mL PBS per well. Then, the rinsed PBS is removed, and 250 μL per well of RIPA Lysis and Extraction Buffer (purchased from Thermo Scientific, 89900) is added to lysis the cells. The obtained solution is collected in a 1.5 mL tube and centrifuged at 300 g for 1 minute. 200 μL of the supernatant is loaded in a 96-well black plate, and the fluorescence is measured at OD485 (excitation) and OD530 (emission) to analyze the amount of ROS.

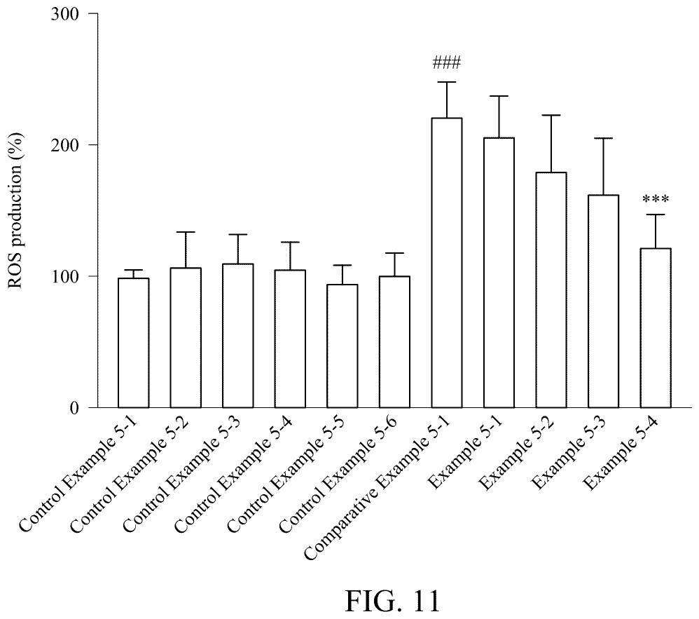

The experimental results for the composition including the extracellular matrix (ECM) and the mitochondria are shown in Table 10 and FIG. 11. FIG. 11 shows the ROS production of the human gingival fibroblasts treated with PM and then treated with the compositions of the examples and the comparative examples, relative to the control group. In FIG. 11, the control group is the cells without PM, mitochondria, and ECM (Control Example 5-1), the symbol “#” represents a statistically significant difference (### indicates P<0.001) relative to the control group (Control Example 5-1), and the symbol “*” represents a statistically significant difference (*** indicates P<0.001) relative to the comparative example (Comparative Example 5-1). From Control Examples 5-1 to 5-6, in the case that the cells are not damaged, the addition of the mitochondria or ECM alone does not affect ROS production, which indicates that the mitochondria and ECM does not induce the human gingival fibroblasts to produce ROS. In addition, from the examples and the comparative examples, in the case that the cells are damaged, the addition of the mitochondria can reduce ROS production (Examples 5-1 and 5-2), which indicates that the addition of the mitochondria contributes to mitigating, repairing, ameliorating, or treating the damage to the human gingival fibroblasts caused by PM, and reduces the damage further caused by ROS. Further, from the examples and the comparative examples, in the case that the cells are damaged, the addition of the composition including the mitochondria and ECM can further reduce ROS production compared to the addition of an equivalent amount of the mitochondria (Examples 5-3 and 5-4) and have a statistically significant difference, which indicates the addition of the composition including the mitochondria and ECM exhibits a synergistic effect in mitigating, repairing, ameliorating, or treating the damage to the human gingival fibroblasts, and can significantly reduce the damage further caused by ROS.

TABLE 10

| | | | | | ROS |
|---|---|---|---|---|---|
| | PM | Mitochondria | | ECM | production |
| Group | μg/cm² | μg | μg/mL | μg/mL | % |
| Control | — | — | — | — | 100 ± 4.8 |
| Example 5-1 | | | | | |
| Control | | 15 | 60 | — | 107.9 ± 26.0 |
| Example 5-2 | | | | | |
| Control | | 40 | 160 | — | 110.8 ± 20.6 |
| Example 5-3 | | | | | |
| Control | | — | — | 15 | 106.7 ± 19.2 |
| Example 5-4 | | | | | |
| Control | | 15 | 60 | 15 | 95.6 ± 12.8 |
| Example 5-5 | | | | | |
| Control | | 40 | 160 | 15 | 101.9 ± 16.2 |
| Example 5-6 | | | | | |
| Comparative | 50 | — | — | — | 222.1 ± 25.8 |
| Example 5-1 | | | | | |
| Example 5-1 | | 15 | 60 | — | 206.7 ± 30.3 |
| Example 5-2 | | 40 | 160 | — | 180.8 ± 41.6 |
| Example 5-3 | | 15 | 60 | 15 | 163.6 ± 41.4 |
| Example 5-4 | | 40 | 160 | 15 | 122.7 ± 24.2 |

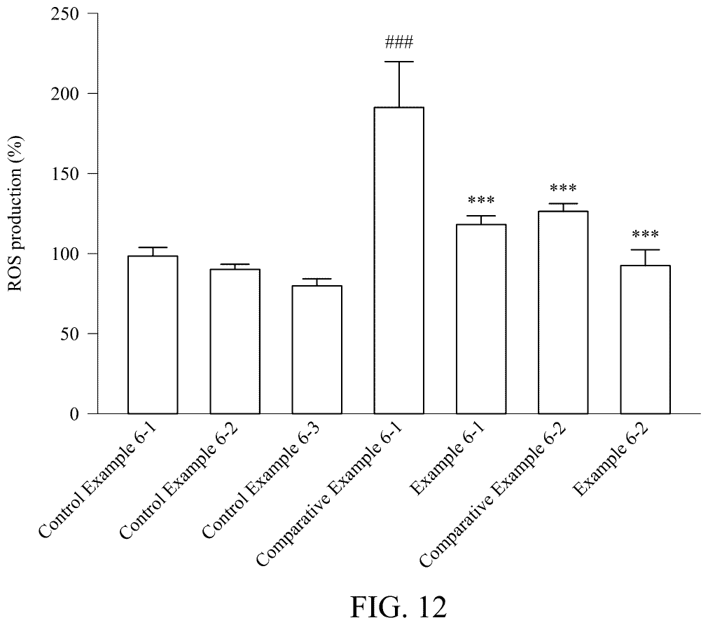

The experimental results for the composition including the PRP-derived extracellular vesicles (PRP-EVs) and the mitochondria are shown in Table 11 and FIG. 12. FIG. 12 shows the ROS production of the human gingival fibroblasts treated with PM and then treated with the compositions of the examples and the comparative examples, relative to the control group. In FIG. 12, the control group is the cells without PM, mitochondria, and PRP-EVs (Control Example 6-1), the symbol “#” represents a statistically significant difference (### indicates P<0.001) relative to the control group (Control Example 6-1), and the symbol “*” represents a statistically significant difference (*** indicates P<0.001) relative to the comparative example (Comparative Example 6-1). From Control Examples 6-1 to 6-3, in the case that the cells are not damaged, the addition of the mitochondria or PRP-EVs alone does not affect ROS production, which indicates that the mitochondria and PRP-EVs does not induce the human gingival fibroblasts to produce ROS. In addition, from the examples and the comparative examples, in the case that the cells are damaged, the addition of the mitochondria can reduce ROS production (Example 6-1), which indicates that the addition of the mitochondria contributes to mitigating, repairing, ameliorating, or treating the damage to the human gingival fibroblasts caused by PM, and reduces the damage further caused by ROS. Further, from the examples and the comparative examples, in the case that the cells are damaged, the addition of the composition including the mitochondria and PRP-EVs can further reduce ROS production compared to the addition of an equivalent amount of the mitochondria (Example 6-2) and have a statistically significant difference, which indicates the addition of the composition including the mitochondria and PRP-EVs exhibits a synergistic effect in mitigating, repairing, ameliorating, or treating the damage to the human gingival fibroblasts, and can significantly reduce the damage further caused by ROS.

TABLE 11

| | | | | | ROS |
|---|---|---|---|---|---|
| | PM | Mitochondria | | PRP-EVs | production |
| Group | μg/cm² | μg | μg/mL | v/v % | % |
| Control | — | — | — | — | 100 ± 3.8 |
| Example 6-1 | | | | | |
| Control | | 40 | 160 | — | 91.9 ± 1.4 |
| Example 6-2 | | | | | |
| Control | | — | — | 2.5 | 81.4 ± 2.9 |
| Example 6-3 | | | | | |
| Comparative | 50 | — | — | — | 193.2 ± 26.7 |
| Example 6-1 | | | | | |
| Example 6-1 | | 40 | 160 | — | 119.7 ± 4.1 |
| Comparative | | — | — | 2.5 | 128.2 ± 3.1 |
| Example 6-2 | | | | | |
| Example 6-2 | | 40 | 160 | 2.5 | 94.3 ± 8.4 |

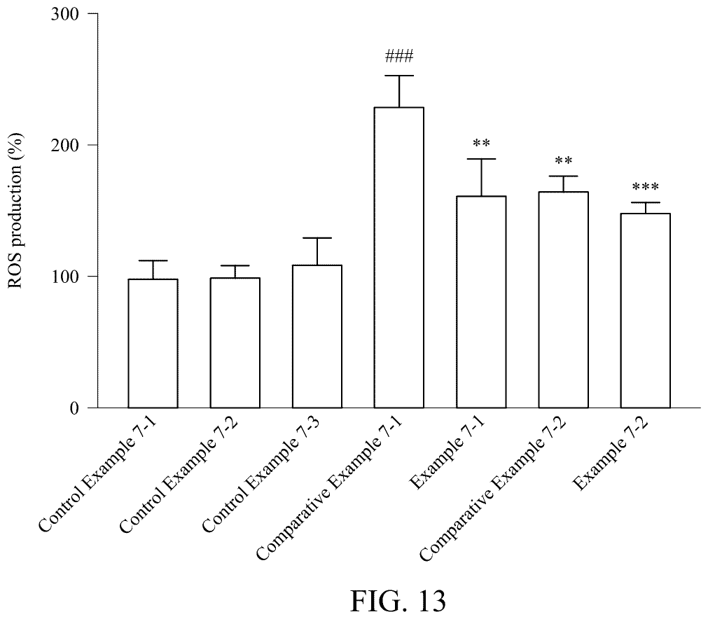

The experimental results for the composition including the MSC-derived extracellular vesicles (MSC-EVs) and the mitochondria are shown in Table 12 and FIG. 13. FIG. 13 shows the ROS production of the human gingival fibroblasts treated with PM and then treated with the compositions of the examples and the comparative examples, relative to the control group. In FIG. 13, the control group is the cells without PM, mitochondria, and MSC-EVs (Control Example 7-1), the symbol “#” represents a statistically significant difference (### indicates P<0.001) relative to the control group (Control Example 7-1), and the symbol “*” represents a statistically significant difference (** indicates P<0.01, and *** indicates P<0.001) relative to the comparative example (Comparative Example 7-1). From Control Examples 7-1 to 7-3, in the case that the cells are not damaged, the addition of the mitochondria or MSC-EVs alone does not affect ROS production, which indicates that the mitochondria and MSC-EVs does not induce the human gingival fibroblasts to produce ROS. In addition, from the examples and the comparative examples, in the case that the cells are damaged, the addition of the mitochondria can reduce ROS production (Example 7-1), which indicates that the addition of the mitochondria contributes to mitigating, repairing, ameliorating, or treating the damage to the human gingival fibroblasts caused by PM, and reduces the damage further caused by ROS. Further, from the examples and the comparative examples, in the case that the cells are damaged, the addition of the composition including the mitochondria and MSC-EVs can further reduce ROS production compared to the addition of an equivalent amount of the mitochondria (Example 7-2) and have a statistically significant difference, which indicates the addition of the composition including the mitochondria and MSC-EVs exhibits a synergistic effect in mitigating, repairing, ameliorating, or treating the damage to the human gingival fibroblasts, and can significantly reduce the damage further caused by ROS.

TABLE 12

| | | | | | ROS |
|---|---|---|---|---|---|
| | PM | Mitochondria | | MSC-EVs | production |
| Group | μg/cm² | μg | μg/mL | μg/mL | % |
| Control | — | — | — | — | 100 ± 12.0 |
| Example 7-1 | | | | | |
| Control | | 40 | 160 | — | 100.8 ± 7.6 |
| Example 7-2 | | | | | |
| Control | | — | — | 10 | 110.7 ± 18.6 |
| Example 7-3 | | | | | |
| Comparative | 50 | — | — | — | 230.6 ± 21.7 |
| Example 7-1 | | | | | |
| Example 7-1 | | 40 | 160 | — | 162.9 ± 26.6 |
| Comparative | | — | — | 10 | 166.1 ± 10.2 |
| Example 7-2 | | | | | |
| Example 7-2 | | 40 | 160 | 10 | 149.5 ± 6.8 |

### Experiment 9: Mitochondria Reducing the Damage to the Mitochondria in the Human Gingival Fibroblasts Caused by PM—Membrane Potential Analysis

The procedure in this experiment is generally the same as that in Experiment 1, and only the differences are described below. PM is added at a concentration of 0 or 50 μg/cm² in the well. After the cells are cultured with PM at 37° C. and 5% CO₂ for 6 hours, the supernatant in the well is removed, and the cells are rinsed with 0.5 mL PBS per well. Then, the rinsed PBS is removed, and a fresh DMEM with 1% FBS (250 μL/well) and the composition of each of the examples and comparative examples are added to react at 37° C. and 5% CO₂ for 20 hours. After cell culture, the supernatant in the well is removed, and the cells are rinsed with 0.5 mL PBS per well. Then, the rinsed PBS is removed, and a fresh DMEM with 1% FBS and 5 μM JC-1 (250 μL/well) is added to react at 37° C. for 10 minutes. After reaction, the supernatant in the well is removed, and the cells are rinsed with 0.5 mL PBS per well twice. Then, a fresh DMEM with 1% FBS (250 μL/well) is added. The fluorescence of JC-1 aggregate is measured at OD520 (excitation) and OD590 (emission), and the fluorescence of JC-1 monomer is measured at OD490 (excitation) and OD530 (emission), thereby evaluating the mitochondrial membrane potential of the human gingival fibroblasts.

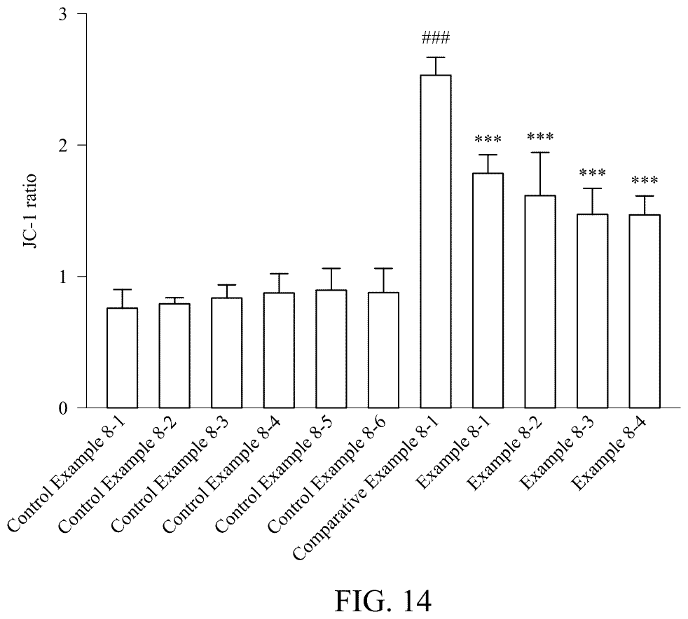

The experimental results for the composition including the extracellular matrix (ECM) and the mitochondria are shown in Table 13 and FIG. 14. FIG. 14 shows the ratio of JC-1 monomer/JC-1 aggregate (JC-1 ratio) of the mitochondria in the human gingival fibroblasts treated with PM and then treated with the compositions of the examples and the comparative examples. In FIG. 14, the control group is the cells without PM, mitochondria, and ECM (Control Example 8-1), the symbol “#” represents a statistically significant difference (### indicates P<0.001) relative to the control group (Control Example 8-1), and the symbol “*” represents a statistically significant difference (*** indicates P<0.001) relative to the comparative example (Comparative Example 8-1). From Control Examples 8-1 to 8-6, in the case that the cells are not damaged, the addition of the mitochondria or ECM alone does not affect the ratio of JC-1 monomer/JC-1 aggregate (JC-1 ratio) of the mitochondria in the human gingival fibroblasts, which indicates that the mitochondria and ECM have no adverse effect on the mitochondrial function of the human gingival fibroblasts. In addition, from the examples and the comparative examples, in the case that the cells are damaged, the addition of the mitochondria can decrease the JC-1 ratio (Examples 8-1 and 8-2), which indicates that the damaged mitochondrial membrane of the human gingival fibroblasts is improved, and further indicates that the addition of the mitochondria can reduce the damage to the mitochondria in the human gingival fibroblasts caused by PM and improve the mitochondrial function of the human gingival fibroblasts. Further, from the examples and the comparative examples, in the case that the cells are damaged, the addition of the composition including the mitochondria and ECM can further decrease the JC-1 ratio compared to the addition of an equivalent amount of the mitochondria (Examples 8-3 and 8-4) and have a statistically significant difference, which indicates the addition of the composition including the mitochondria and ECM exhibits a synergistic effect in mitigating, repairing, ameliorating, or treating the damage to the human gingival fibroblasts, and can further improve the mitochondrial function of the human gingival fibroblasts.

TABLE 13

| | PM | Mitochondria | | ECM | JC-1 ratio |
|---|---|---|---|---|---|
| Group | μg/cm² | μg | μg/mL | μg/mL | — |
| Control | — | — | — | — | 0.77 ± 0.12 |
| Example 8-1 | | | | | |
| Control | | 15 | 60 | — | 0.81 ± 0.03 |
| Example 8-2 | | | | | |
| Control | | 40 | 160 | — | 0.85 ± 0.09 |
| Example 8-3 | | | | | |
| Control | | — | — | 15 | 0.89 ± 0.13 |
| Example 8-4 | | | | | |
| Control | | 15 | 60 | 15 | 0.91 ± 0.15 |
| Example 8-5 | | | | | |
| Control | | 40 | 160 | 15 | 0.89 ± 0.17 |
| Example 8-6 | | | | | |
| Comparative | 50 | — | — | — | 2.55 ± 0.12 |
| Example 8-1 | | | | | |
| Example 8-1 | | 15 | 60 | — | 1.80 ± 0.13 |
| Example 8-2 | | 40 | 160 | — | 1.63 ± 0.31 |
| Example 8-3 | | 15 | 60 | 15 | 1.49 ± 0.18 |
| Example 8-4 | | 40 | 160 | 15 | 1.49 ± 0.12 |

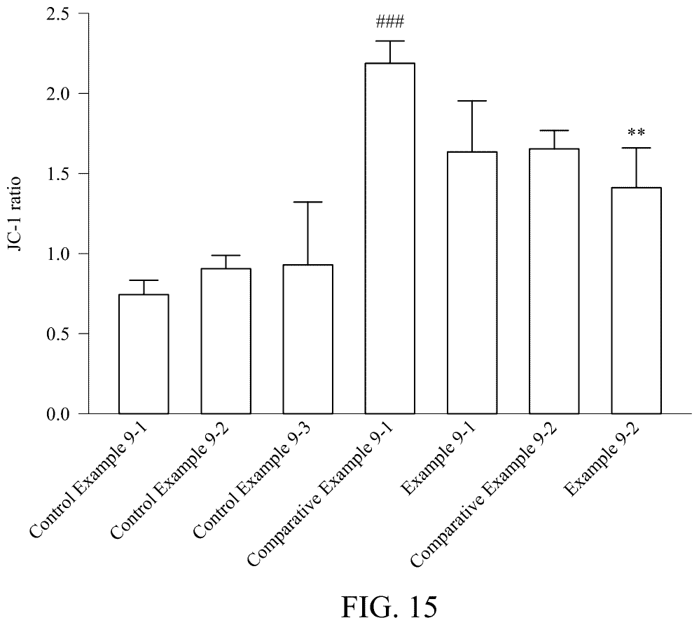

The experimental results for the composition including the PRP-derived extracellular vesicles (PRP-EVs) and the mitochondria are shown in Table 14 and FIG. 15. FIG. 15 shows the ratio of JC-1 monomer/JC-1 aggregate (JC-1 ratio) of the mitochondria in the human gingival fibroblasts treated with PM and then treated with the compositions of the examples and the comparative examples. In FIG. 15, the control group is the cells without PM, mitochondria, and PRP-EVs (Control Example 9-1), the symbol “#” represents a statistically significant difference (### indicates P<0.001) relative to the control group (Control Example 9-1), and the symbol “*” represents a statistically significant difference (** indicates P<0.01) relative to the comparative example (Comparative Example 9-1). From Control Examples 9-1 to 9-3, in the case that the cells are not damaged, the addition of the mitochondria or RP-EVs alone does not affect the ratio of JC-1 monomer/JC-1 aggregate (JC-1 ratio) of the mitochondria in the human gingival fibroblasts, which indicates that the mitochondria and PRP-EVs have no adverse effect on the mitochondrial function of the human gingival fibroblasts. In addition, from the examples and the comparative examples, in the case that the cells are damaged, the addition of the mitochondria can decrease the JC-1 ratio (Example 9-1), which indicates that the damaged mitochondrial membrane of the human gingival fibroblasts is improved, and further indicates that the addition of the mitochondria can reduce the damage to the mitochondria in the human gingival fibroblasts caused by PM and improve the mitochondrial function of the human gingival fibroblasts. Further, from the examples and the comparative examples, in the case that the cells are damaged, the addition of the composition including the mitochondria and PRP-EVs can further decrease the JC-1 ratio compared to the addition of an equivalent amount of the mitochondria (Example 9-2) and have a statistically significant difference, which indicates the addition of the composition including the mitochondria and PRP-EVs exhibits a synergistic effect in mitigating, repairing, ameliorating, or treating the damage to the human gingival fibroblasts, and can further improve the mitochondrial function of the human gingival fibroblasts.

TABLE 14

| | PM | Mitochondria | | PRP-EVs | JC-1 ratio |
|---|---|---|---|---|---|
| Group | μg/cm² | μg | μg/mL | v/v % | — |
| Control | — | — | — | — | 0.76 ± 0.08 |
| Example 9-1 | | | | | |
| Control | | 40 | 160 | — | 0.92 ± 0.07 |
| Example 9-2 | | | | | |
| Control | | — | — | 2.5 | 0.95 ± 0.38 |
| Example 9-3 | | | | | |
| Comparative | 50 | — | — | — | 2.20 ± 0.12 |
| Example 9-1 | | | | | |
| Example 9-1 | | 40 | 160 | — | 1.65 ± 0.30 |
| Comparative | | — | — | 2.5 | 1.67 ± 0.1 |
| Example 9-2 | | | | | |
| Example 9-2 | | 40 | 160 | 2.5 | 1.43 ± 0.23 |

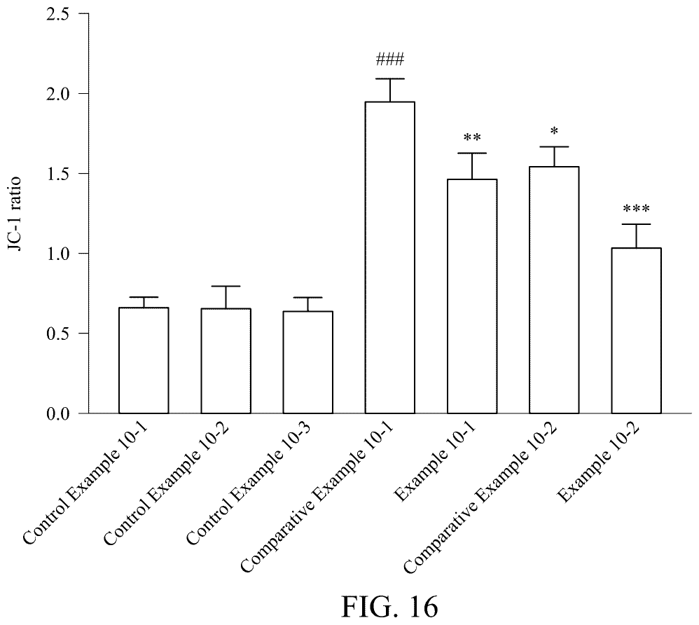

The experimental results for the composition including the MSC-derived extracellular vesicles (MSC-EVs) and the mitochondria are shown in Table 15 and FIG. 16. FIG. 16 shows the ratio of JC-1 monomer/JC-1 aggregate (JC-1 ratio) of the mitochondria in the human gingival fibroblasts treated with PM and then treated with the compositions of the examples and the comparative examples. In FIG. 16, the control group is the cells without PM, mitochondria, and MSC-EVs (Control Example 10-1), the symbol “#” represents a statistically significant difference (### indicates P<0.001) relative to the control group (Control Example 10-1), and the symbol “*” represents a statistically significant difference (* indicates P<0.05, ** indicates P<0.01, and *** indicates P<0.001) relative to the comparative example (Comparative Example 10-1). From Control Examples 10-1 to 10-3, in the case that the cells are not damaged, the addition of the mitochondria or MSC-EVs alone does not affect the ratio of JC-1 monomer/JC-1 aggregate (JC-1 ratio) of the mitochondria in the human gingival fibroblasts, which indicates that the mitochondria and MSC-EVs have no adverse effect on the mitochondrial function of the human gingival fibroblasts. In addition, from the examples and the comparative examples, in the case that the cells are damaged, the addition of the mitochondria can decrease the JC-1 ratio (Example 10-1), which indicates that the damaged mitochondrial membrane of the human gingival fibroblasts is improved, and further indicates that the addition of the mitochondria can reduce the damage to the mitochondria in the human gingival fibroblasts caused by PM and improve the mitochondrial function of the human gingival fibroblasts. Further, from the examples and the comparative examples, in the case that the cells are damaged, the addition of the composition including the mitochondria and MSC-EVs can further decrease the JC-1 ratio compared to the addition of an equivalent amount of the mitochondria (Example 10-2) and have a statistically significant difference, which indicates the addition of the composition including the mitochondria and MSC-EVs exhibits a synergistic effect in mitigating, repairing, ameliorating, or treating the damage to the human gingival fibroblasts, and can further improve the mitochondrial function of the human gingival fibroblasts.

TABLE 15

| | PM | Mitochondria | | MSC-EVs | JC-1 ratio |
|---|---|---|---|---|---|
| Group | μg/cm² | μg | μg/mL | μg/mL | — |
| Control | — | — | — | — | 0.68 ± 0.05 |
| Example 10-1 | | | | | |
| Control | | 40 | 160 | — | 0.67 ± 0.12 |
| Example 10-2 | | | | | |
| Control | | — | — | 10 | 0.65 ± 0.07 |
| Example 10-3 | | | | | |
| Comparative | 50 | — | — | — | 1.96 ± 0.13 |
| Example 10-1 | | | | | |
| Example 10-1 | | 40 | 160 | — | 1.48 ± 0.15 |
| Comparative | | — | — | 10 | 1.56 ± 0.11 |
| Example 10-2 | | | | | |
| Example 10-2 | | 40 | 160 | 10 | 1.05 ± 0.13 |

### Experiment 10: Mitochondria Reducing the Damage to the Mitochondria in the Human Gingival Fibroblasts Caused by PM—ATP Production

The procedure in this experiment is generally the same as that in Experiment 1, and only the differences are described below. The human gingival fibroblasts are cultured at a density of 3.5×10⁵ cells in 10 mL of DMEM with 100% FB S in a 10 cm dish, in which the bottom area of the dish is 60.8 cm², for 24 hours. Then, after the cells are cultured until the dish is 8000 full, the culture medium is removed, and the cells are rinsed with 5 mL PBS per dish. Then, the rinsed PB S is removed, and a fresh DMEM with 10% FB S (5 mL/dish) is added. Then, PM is added at a concentration of 0 or 50 μg/cm^{a} in the dish. After the cells are cultured with PM at 37° C. and 50% CO₂ for 6 hours, the supermatant is removed, and the cells are rinsed with PBS. Then, the rinsed PBS is removed, and a fresh DMVEM with 10% FB S and the composition of each of the examples and comparative examples are added to culture at 37° C. and 50% CO₂ for 20 hours. After cell culture, the ATP production of the mitochondria in the cells is analyzed by using an ATP assay kit.

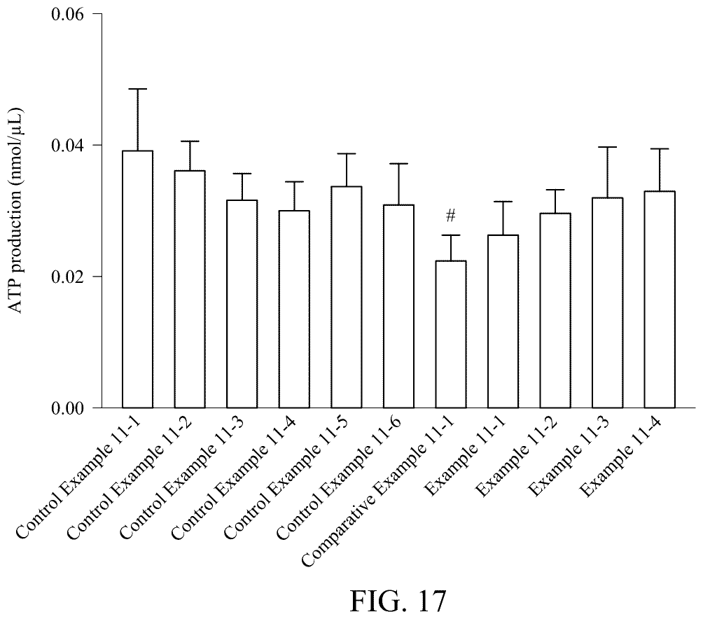

The experimental results are shown in Table 16 and FIG. 17. FIG. 17 shows the ATP production of the mitochondria in the human gingival fibroblasts treated with PM and then treated with the compositions of the examples and the comparative examples. In FIG. 17, the control group is the cells without PM, mitochondria, and ECM (Control Example 11-1), and the symbol “#” represents a statistically significant difference (# indicates P<0.05) relative to the control group (Control Example 11-1). From Control Examples 11-1 to 11-6, in the case that the cells are not damaged, the addition of the mitochondria or ECM alone does not affect the ATP production of the mitochondria in the human gingival fibroblasts, which indicates that the mitochondria and ECM have no adverse effect on the ATP-production capacity of the mitochondria in the human gingival fibroblasts. In addition, from the examples and the comparative examples, in the case that the cells are damaged, the addition of the mitochondria can increase the mitochondrial ATP production of the human gingival fibroblasts (Examples 11-1 and 11-2), which indicates that the ATP-production capacity of the damaged mitochondria in the human gingival fibroblasts is improved, and further indicates that the addition of the mitochondria can reduce the damage to the mitochondria in the human gingival fibroblasts caused by PM and improve the mitochondrial function of the human gingival fibroblasts. Further, from the examples and the comparative examples, in the case that the cells are damaged, the addition of the composition including the mitochondria and ECM can further increase the mitochondrial ATP production of the human gingival fibroblasts compared to the addition of an equivalent amount of the mitochondria (Examples 11-3 and 11-4), which indicates the addition of the composition including the mitochondria and ECM exhibits a synergistic effect in mitigating, repairing, ameliorating, or treating the damage to the human gingival fibroblasts, and can further improve the mitochondrial function of the human gingival fibroblasts.

TABLE 16

| | PM | Mitochondria | | ECM | ATP production |
|---|---|---|---|---|---|
| Group | μg/cm² | μg | μg/mL | μg/mL | nmol/μL |
| Control | — | — | — | — | 0.039 ± 0.009 |
| Example 11-1 | | | | | |
| Control | | 15 | 60 | — | 0.036 ± 0.004 |
| Example 11-2 | | | | | |
| Control | | 40 | 160 | — | 0.032 ± 0.004 |
| Example 11-3 | | | | | |
| Control | | — | — | 15 | 0.030 ± 0.004 |
| Example 11-4 | | | | | |
| Control | | 15 | 60 | 15 | 0.034 ± 0.005 |
| Example 11-5 | | | | | |
| Control | | 40 | 160 | 15 | 0.031 ± 0.006 |
| Example 11-6 | | | | | |
| Comparative | 50 | — | — | — | 0.023 ± 0.004 |
| Example 11-1 | | | | | |
| Example 11-1 | | 15 | 60 | — | 0.027 ± 0.005 |
| Example 11-2 | | 40 | 160 | — | 0.030 ± 0.003 |
| Example 11-3 | | 15 | 60 | 15 | 0.032 ± 0.007 |
| Example 11-4 | | 40 | 160 | 15 | 0.033 ± 0.006 |

According to the above experiments and the embodiments of the present disclosure, the composition including mitochondria may reduce the damage caused by particulate matters to gingival fibroblasts, thereby reducing the death of gingival fibroblasts. Also, the composition including mitochondria may reduce the aging of gingival fibroblasts induced by particulate matters. Also, the composition including mitochondria may reduce the production of reactive oxygen species (ROS) in gingival fibroblasts induced by particulate matters, thereby reducing the further damage caused by the reactive oxygen species to gingival fibroblasts. In addition, the composition including mitochondria may reduce the damage caused by particulate matters to the mitochondria in gingival fibroblasts, thereby improving the mitochondrial function of gingival fibroblasts. Further, the composition including mitochondria and platelet-rich plasma-derived extracellular vesicles, the composition including mitochondria and stem cell-derived extracellular vesicles, and the composition including mitochondria and extracellular matrix exhibit synergistic effects in mitigating, repairing, ameliorating, or treating the damage to gingival fibroblasts, significantly reduce the aging or death of gingival fibroblasts caused by particulate matters, further reduce the production of reactive oxygen species and the associated damage, and further improve the mitochondrial function of gingival fibroblasts. Therefore, the composition of the embodiments of the present disclosure may achieve the purposes of mitigating, repairing, ameliorating, or treating oral damage, and may be expected to be a composition or medicament that is able to mitigate, repair, ameliorate or treat periodontal disease, periodontal abscess, oral submucous fibrosis, Leukoplakia or oral cancer while having both safety and effectiveness.

What is claimed is:

1. A use of mitochondria in manufacturing a composition for oral damage mitigation.

2. The use of claim 1, wherein the oral damage comprises periodontal disease, periodontal abscess, oral submucous fibrosis, Leukoplakia or oral cancer.

3. The use of claim 1, wherein the oral damage mitigation comprises reducing the death of gingival fibroblasts.

4. The use of claim 1, wherein the oral damage mitigation comprises reducing the aging of gingival fibroblasts.

5. The use of claim 1, wherein the oral damage mitigation comprises reducing reactive oxygen species produced by gingival fibroblasts.

6. The use of claim 1, wherein the oral damage mitigation comprises improving the mitochondrial function of gingival fibroblasts.

7. The use of claim 1, wherein the oral damage mitigation comprises improving the mitochondrial membrane potential of gingival fibroblasts or improving the mitochondrial ATP production of gingival fibroblasts.

8. The use of claim 1, wherein an effective dose of the mitochondria in the composition is at least 15 μg.

9. The use of claim 1, wherein the composition further comprises extracellular vesicles or extracellular matrix.

10. The use of claim 9, wherein the extracellular vesicles are derived from platelet-rich plasma (PRP) or stem cells.

11. The use of claim 10, wherein the stem cells are mesenchymal stem cells.

12. The use of claim 10, wherein the mitochondria and the extracellular vesicles are derived from the same stem cells.

13. A composition, comprising mitochondria and a biocompatible carrier.

14. The composition of claim 13, wherein an effective dose of the mitochondria in the composition is at least 15 μg.

15. The composition of claim 13, further comprising extracellular vesicles or extracellular matrix.

16. The composition of claim 15, wherein the extracellular vesicles are derived from platelet-rich plasma (PRP) or stem cells.

17. The composition of claim 16, wherein the stem cells are mesenchymal stem cells.

18. The composition of claim 16, wherein the mitochondria and the extracellular vesicles are derived from the same stem cells.

19. A method for manufacturing a composition comprising mitochondria and extracellular vesicles, comprising:
culturing cells with culture medium in a container;
separating supernatant in the container from the cells adhering to the container after culturing;
collecting extracellular vesicles from the supernatant;
lysing the cells to isolate mitochondria inside the cells; and
mixing the extracellular vesicles and the mitochondria to obtain the composition.

20. The method of claim 19, wherein the mitochondria and the extracellular vesicles are derived from the same cells.
